(19) United States
(12) Patent Application Publication
(10) Pub. No.: US 2007/0087042 A1
(43) Pub. Date: Apr. 19, 2007

(54) A SHROUD FOR SURROUNDING A PROBLEM TISSUE FOR PROMOTING HEALING OF PROBLEM TISSUE ADJACENT HEALTHY SKIN

(76) Inventor: Mark Todd Thomas, Kettering, OH (US)

Correspondence Address: A PATENT LAWYER CORP, PC;R WILLIAM GRAHAM, 22 S ST CLAIR ST, DAYTON, OH, 45402, US

(21) Appl. No.: 11/163,374

(22) Filed: Oct. 17, 2005

(51) Int. Cl. A61L 15/00

(52) U.S. Cl. 424/445; 602/1

(57) ABSTRACT

A shroud for disposal on healthy skin tissue surrounding a problem tissue and for promoting one of an open-closed environment around the problem tissue includes a relatively rigid plastic member having an outer peripheral flange surface configured for disposal adjacent the skin surrounding the problem tissue, wherein the flange can be provided with an adhesive material which preferably extends along the length of the flange to secure the shroud to the healthy skin tissue, a side extends generally lateral from the flange and a top surface panel interconnects the side and provides a non-contacting cover for the problem tissue, and an open-close portion is provided such that when the shroud is disposed adjacent the healthy skin tissue, the open-close portion can either permit the problem tissue to be exposed to air or not.

### BACKGROUND OF THE INVENTION

1. Field of the Invention

The invention relates to promoting healthy tissue and/or ridding problem tissue. More particularly, the invention relates to promoting healing of problem tissue by creating a shroud surrounding the problem tissue which promotes healthy tissue.

2. Description of the Prior Art

It is known that providing oxygen or air to a skin wound such as a cut, a sore or ulcer promotes healing of the wound. In a hospital environment, expensive equipment is used to provide a stream of oxygen onto the wound while keeping the wound free from contaminants. For example, hyperbaric chambers have been used in some circumstances where the wound is severe. Most skin wounds are currently treated with a bandage or stitches. In other circumstances, such as in the case of mole removal, the patient is lanced to remove an area of tissue surrounding the mole and the surface is left exposed. The patient is advised that the area must be kept free from water for a period of 24 hours and thus is unable to shower or bath during such period. Further, it is preferred that the lanced area remain open to air and avoid contact with clothing or the like.

Further, other types of skin ailments such as plantar warts, corns and the like are typically treated with a compound to remove the ailment wherein the aliment is covered in a manner to prevent air from contacting the ailment. This is the case with plantar wart where tape is used to cover the wart after the removal compound is applied. However, such type wart is difficult to remove as the wart tends to be pushed back into the foot and embeds itself into the foot tissue. Ring-like adhesive tape has been made as an attempt to keep the wart from pushing inward. However, such existing tape has not proven effective.

There remains a need for a convenient means for treating various types of minor skin wounds and afflictions in a relatively inexpensive manner. It would therefore be of significant value to have a device which provides a protective area about a skin wound or ailment having a dual capability to enable both an open and closed environment about the wound or ailment. The present invention overcomes the deficiencies in the art by providing such a device.

### SUMMARY OF THE INVENTION

It is an object to promote healthy tissue and treat problem tissue of a human or animal.

It is an object to ease removal of unhealthy tissue on a human or animal.

It is an object to provide shroud for use on skin to surround a wound or other aliment which can be selectively openable or closable to permit air exchange to the wound or ailment.

It is an object to provide shroud for use on skin to surround a wound or other aliment which is relatively rigid and configured to prevent contact with the wound or ailment.

It is an object to protect a wound or ailing tissue from contacting clothing.

It is an object to provide shroud for use on skin to surround a wound or other aliment which is disposable.

It is an object to provide shroud for use on skin to surround a problem tissue which is relatively comfortable and simple to use.

Accordingly, the present invention is directed to a shroud for disposal on healthy skin tissue surrounding a problem tissue and for promoting one of an open/closed environment around the problem tissue. The shroud includes a relatively rigid plastic member having an outer peripheral flange surface configured for disposal adjacent the skin surrounding the problem tissue. The flange can be provided with an adhesive material which preferably extends along the length of the flange to secure the shroud to the healthy skin tissue. The shroud includes a side which extends generally lateral from the flange and a top surface panel which interconnect the side.

In one embodiment, the shroud is generally circular in shape and the flange, side and top surface panel are integrally formed. The top surface panel is laterally displaced from the flange and when the flange is connected to the skin, the top surface panel is laterally displaced from the wound or ailment to provide a protective non-contacting cover. The shroud can preferably be constructed rigid enough to substantially prevent undue bending of the shroud when pressure of one's body or clothes is applied to the shroud whereby such pressure is distributed onto healthy skin tissue upon which the flange is disposed. The shroud can include an open/close portion (which can be an integral or separate on off part) such that when the shroud is disposed adjacent the healthy skin tissue, the open/close portion can either permit the problem tissue to be exposed to air or not.

The top surface panel can be formed with a tab (or cap) which is configured to sealably snap fit to a tab (or cap) retention surface which forms an opening on the top surface panel. In another embodiment, the top surface panel can be formed with one or more cut-outs and a cover slidably connects to the top surface panel to slide over the cut-outs to close the shroud. In yet another embodiment, the top surface panel includes a raised mesh surface and a cap which connects about the mesh surface to seal the shroud.

### BRIEF DESCRIPTION OF THE DRAWINGS

FIG. 1 is a perspective view of one embodiment of the device of this invention.

FIG. 2 is a cross-sectional view taken on line 2-2 of FIG. 1 showing the shroud in a closed position.

FIG. 3 is cross-sectional view of the shroud in FIG. 2 showing an open position.

FIG. 4 is a top view illustrating another embodiment of the invention in showing the shroud in a closed position.

FIG. 5 is a side view of the shroud in FIG. 4 an open position.

FIG. 6 is perspective view of the shroud of FIG. 4 shown on a bottom of a foot.

FIG. 7 is perspective view of the shroud in use.

FIG. 8 is a plan view of another embodiment of a shroud of the invention.

FIG. 9 depicts a side sectional view of a cap for use with the embodiment in FIG. 8.

FIG. 10 shows a side sectional view of a part of the shroud in FIG. 8.

FIG. 11 illustrates a perspective view of the shroud of the embodiment in FIG. 8.

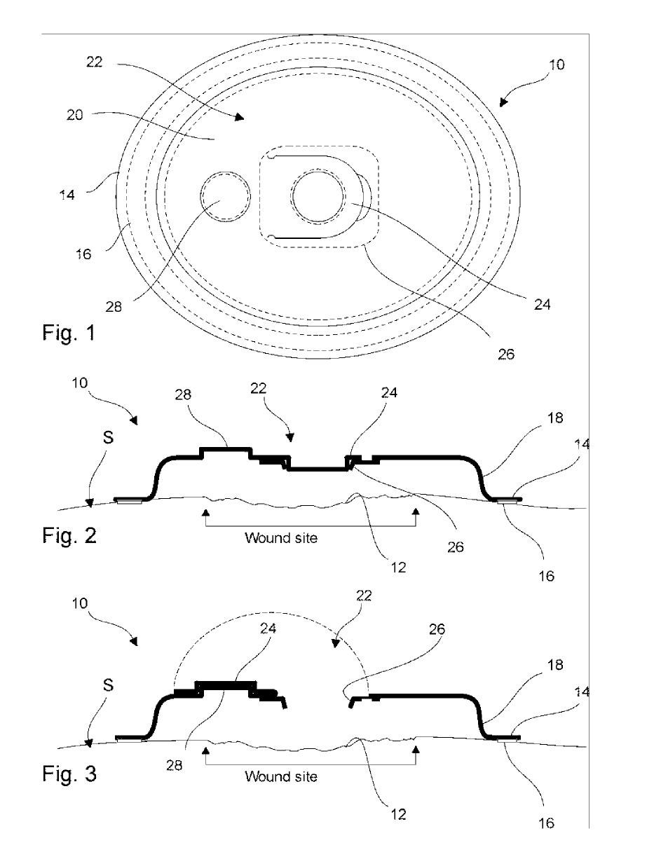

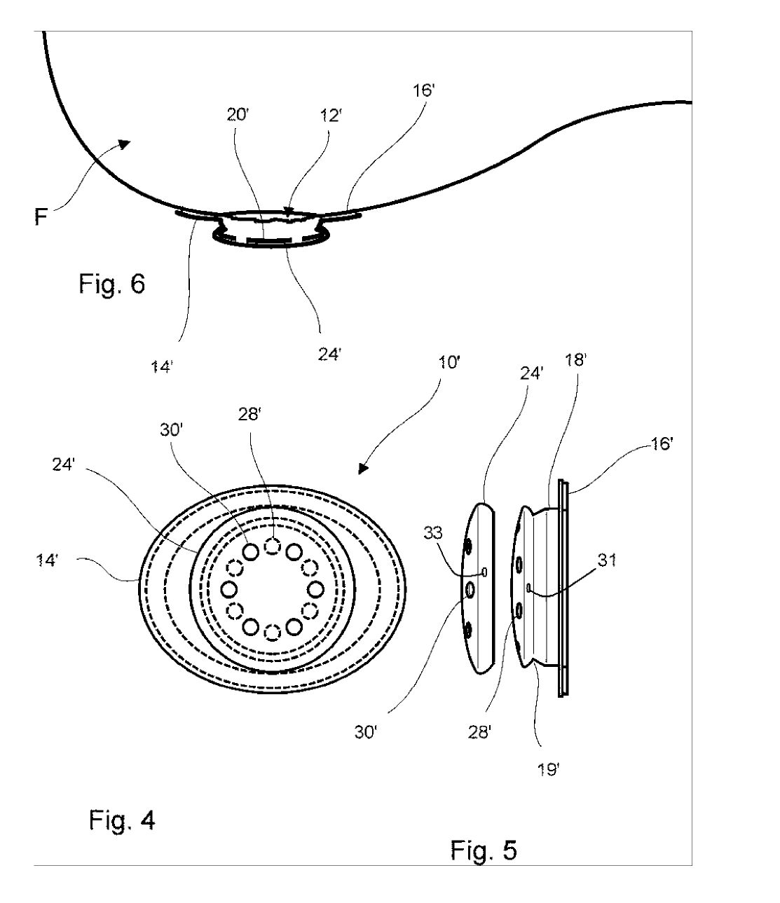

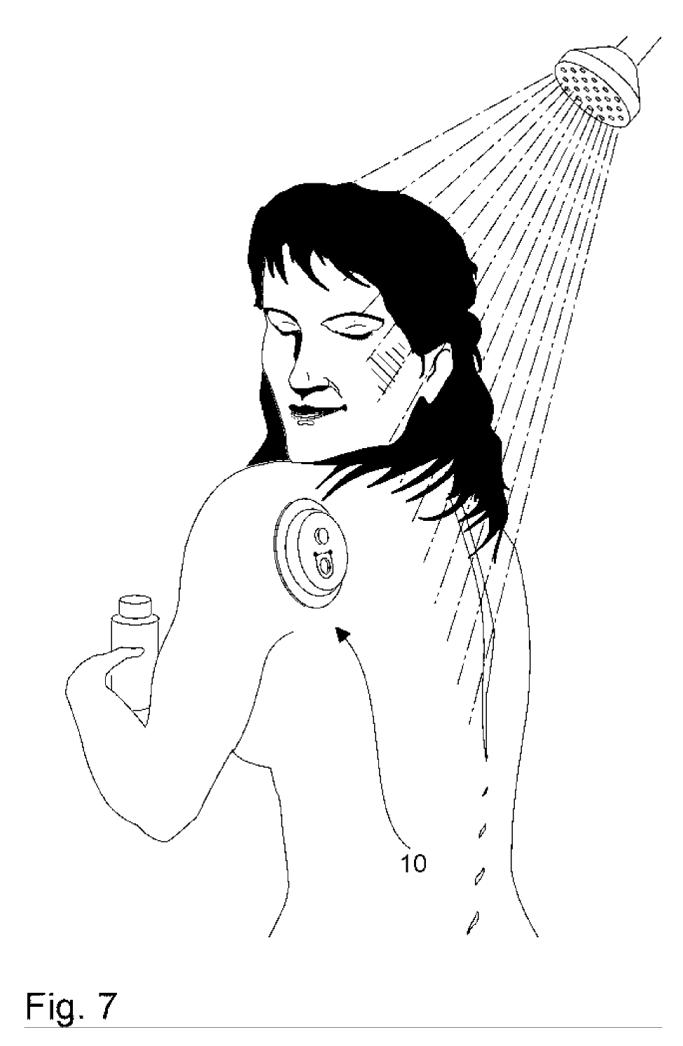

### DETAILED DESCRIPTION AND PREFERRED EMBODIMENTS

Referring now to the drawings, one embodiment is directed to a shroud 10 for surrounding a problem tissue 12 (wound or ailment) for promoting healing and or treating problem tissue 12. The shroud 10 includes a relatively rigid plastic member which can be made of a medical grade material, such as medical grade plastic. The shroud 10 has an outer peripheral flange 14 configured for disposal adjacent healthy skin S surrounding the problem tissue 12.

The flange 14 can be provided with a double sided adhesive tape 16 having a peel off backing which preferably extends along the length of the flange 14 to secure the shroud 10 to the skin S. The adhesive tape 16 is used to secure the shroud 10 to the patient's skin S around the problem tissue 12. The adhesive tape 16 will not touch the problem tissue 12. The adhesive tape 16 can preferably be constructed to be water resistant or waterproof material and disposed between the bottom of the flange 14 and the top of skin S. The adhesive tape 16 can be formed of a foam core permeable to air.

The shroud 10 includes a side 18 which can extend generally lateral and normal from the flange 14 and is here shown as a continuous side integrally connected to the flange 14. A top surface panel 20 interconnects to the side 18 and is likewise integrally formed thereto. In this embodiment, the shroud 10 is shown generally circular or oval in shape and the flange, side and top surface panel are integrally formed. The top surface panel 20 is laterally displaced from the flange 14 and generally parallel thereto such that when the flange 14 is connected to the skin S, the top surface panel 20 is laterally displaced from the problem tissue 12 to provide a protective non-contacting cover over the problem tissue 12.

The shroud 10 can preferably be constructed rigid enough to substantially prevent undue bending of the shroud 10 when pressure of one's body is applied to the shroud 10, such as top surface panel 20, whereby such pressure is distributed onto healthy skin S upon which the flange 14 is disposed. The shroud can include an open/close portion 22 such that when the shroud 10 is disposed adjacent the skin S, the open/close portion 22 can either permit the problem tissue 12 to be exposed to air or not. In this embodiment, the top surface panel 20 can be formed with a tab 24 which is configured to sealably snap fit to a tab surface 26 which forms an opening on the top surface panel 20 to form a closed position or snap fit to a protruding tab surface 28 integrally formed on the top surface panel 20 to form an open position wherein air exchange occurs through the opening in defined by tab surface 26.

In another embodiment, the shroud 10′ differs from shroud 10 in that the top surface panel 20′ can be formed with one or more cut-outs 28′. A snap fit cover 24′ connects to a lip 19′ formed on side 18′ and is slidably disposed on the top surface panel 20′ and includes like formed cut-outs 30′ which can spatially align to slide over the cut-out 28′ to open/close the shroud 10′.

In the case of treating ailments such as a plantar wart 12′, it may be preferable to leave the shroud 10/10′ in the closed position. Here, the top surface panel 20′ and cover 24′ are dome shaped to aid in this regard. The shroud 10/10′ keeps the wart 12′ protruding outward from the bottom of the foot F.

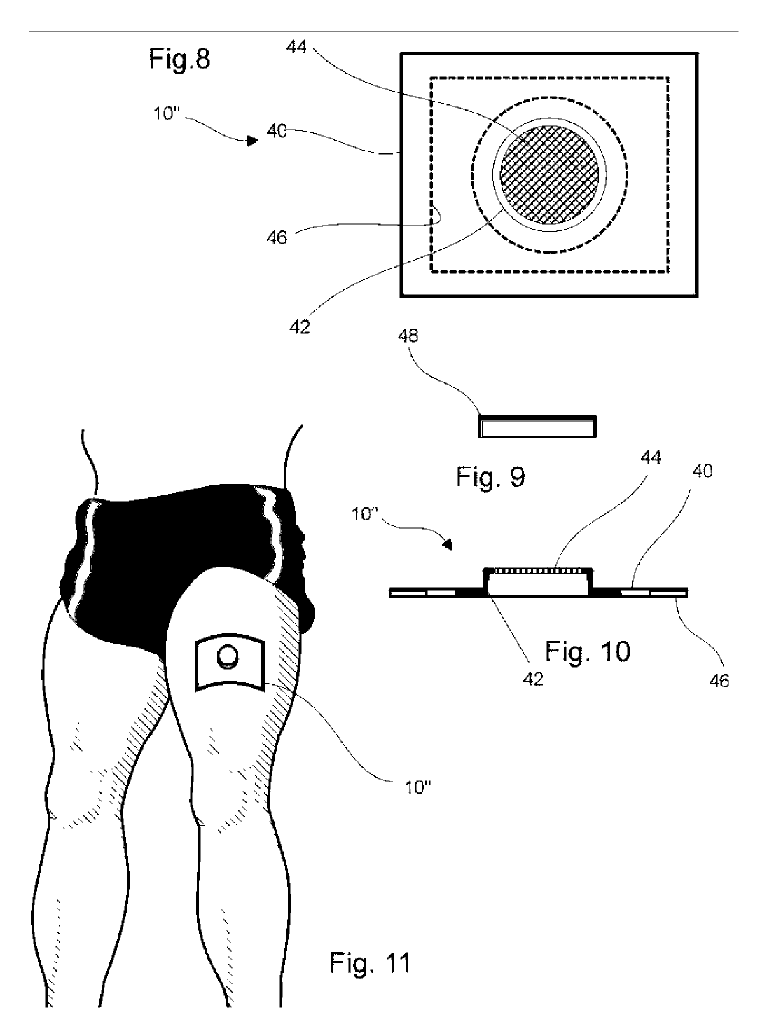

Still another embodiment is shown in FIGS. 8-11 wherein the shroud 10″ includes a base 40, and top surface panel includes a raised mesh portion 44 which is connected to a side 42. An adhesive member 46 can likewise be used to secure the shroud 10″ to the wearer. Also, a cap 48 is provided which is of a size and configuration to snap fit about the side 42 and mesh 44 to seal about the same and prevent dirt or water from entering therethrough.

Thus, in an open one mode of the invention, the open air exchange increases the oxygen surrounding the wound and aids in healing the same. In another closed mode of the invention, the closed environment prevents air/oxygen from reaching the aliment, such as a viral affliction, wart, and aids in the ridding thereof. In either case, the shroud is constructed with a relatively rigid material to distribute the pressure applied thereto onto the flange and in turn healthy skin S which either protects the wound or aids in unseating the wart. Further, the closed position also provides a wearer of the shroud to take a shower or bath without affecting the wound or potentially spreading a virus (e.g., plantar wart).

The shroud of the present invention is designed for easy use and is inexpensive and disposable in nature. It is contemplated that the embodiments may not necessarily be disposable and the adhesive tape could be reapplied.

It is contemplated that the shroud of the instant invention be constructed in a variety of sizes and shapes to conform to different wound and ailment sizes. The shroud can also be of other geometric configurations. A typical size width for the shroud is about ½″-2″ and height of about ¼″.

It will be apparent to those skilled in the art that there are numerous modifications, derivations and improvements within the scope and spirit of the invention. The above description and drawings herewith are therefore intended to be exemplary only not limiting and the scope of the invention are to be determined solely by the claims hereto.

What is claimed is:

1. A shroud for disposal on healthy skin tissue surrounding a problem tissue and for promoting one of an open/closed environment around the problem tissue, which includes a relatively rigid plastic member having an outer peripheral flange configured for disposal adjacent the healthy skin tissue surrounding the problem tissue, a side which extends generally lateral from the flange and a top surface panel which connects to the side, wherein said top surface panel is laterally displaced from said flange and when said flange is connected to the skin, said top surface panel is laterally displaced from said problem tissue to provide a protective non-contacting cover over the problem tissue and said shroud further includes an open/close portion such that when said shroud is disposed adjacent the healthy skin tissue, said open/close portion selectively permits the problem tissue to be exposed to air.

2. The shroud of claim 1, which further includes an adhesive material which extends along said flange to sealably secure said shroud to the healthy skin.

3. The shroud of claim 1, wherein said flange, said side and said top surface panel are integrally formed.

4. The shroud of claim 1, wherein said shroud is constructed rigid enough to substantially prevent undue bending of said shroud when pressure of one's body is applied to said shroud whereby such pressure is distributed onto the healthy skin tissue upon which the flange is disposed.

5. The shroud of claim 2, wherein said open/close portion is closable to substantially prevent water from entering said shroud when attached to the healthy skin tissue.

6. The shroud of claim 5, wherein said top surface panel is formed with a tab which is configured to sealably snap fit to a tab surface which forms an opening on said top surface panel.

7. The shroud of claim 5, wherein said top surface panel can be formed with a cut-out and a cover slidably connects to said top surface panel to slide over said cut-out to close said shroud.

8. The shroud of claim 5, wherein there is formed a cap surface about said open/close portion and said shroud includes a removable cap configured to sealably snap fit to said cap surface.

9. A shroud for disposal on healthy skin tissue surrounding a problem tissue, which includes a relatively rigid plastic member having an outer peripheral flange configured for disposal adjacent the healthy skin tissue surrounding the problem tissue, a side which extends generally lateral from the flange and a top surface panel which connects to the side, wherein said top surface panel is laterally displaced from said flange and when said flange is connected to the skin provides a protective non-contacting cover for the problem tissue and wherein said shroud is constructed rigid enough to substantially prevent undue bending of said shroud when pressure of one's body is applied to said shroud whereby such pressure is distributed onto the healthy skin tissue upon which the flange is disposed.

10. The shroud of claim 9, which further includes an open/close portion such that when said shroud is disposed adjacent the healthy skin, said open/close portion selectively permits the problem tissue to be exposed to air.

11. The shroud of claim 9, which further includes an adhesive material which extends along said flange to sealably secure said shroud to the healthy skin tissue.

12. The shroud of claim 9, wherein said flange, said side and said top surface panel are integrally formed.

13. The shroud of claim 11, wherein said open/close portion is closable to substantially prevent water from entering said shroud when so attached to the healthy skin tissue.

14. The shroud of claim 11, wherein said top surface panel is formed with a tab which is configured to sealably snap fit to a tab surface which forms an opening on said top surface panel.

15. The shroud of claim 10, wherein said top surface panel can be formed with a cut-out and a cover slidably connects to said top surface panel to slide over said cut-out to close said shroud.

16. The shroud of claim 10, wherein there is formed a cap surface about said open/close portion and said shroud includes a removable cap configured to sealably snap fit to said cap surface.
